(19) United States
(12) Patent Application Publication
(10) Pub. No.: US 2003/0134352 A1
(43) Pub. Date: Jul. 17, 2003

(54) Facilitating protein folding and solubility by use of peptide extensions

(76) Inventors: Paul I. Freimuth, East Setauket, NY (US); Yian-Biao Zhang, Middle Island, NY (US); Jason A. Howitt, Wading River, NY (US)

Correspondence Address: Margaret C. Bogosian, Brookhaven National Laboratory, Bldg. 475D, P.O. Box 5000, Upton, NY, 11973-5000, US

(21) Appl. No.: 10/037,243

(22) Filed: Jan. 4, 2002

(51) Int. Cl. C12P 21/02; C12N 5/06; C07H 21/04; C12N 15/85

(52) U.S. Cl. 435/69.1; 435/455; 435/320.1; 435/325; 536/23.2

(57) ABSTRACT

Disclosed herein are novel compositions and methods for enhancing the solubility and promoting the adoption of native folding conformation of a protein or polypeptide expressed by recombinant DNA techniques. One embodiment of the present invention relates to a protein or polypeptide of interest is modified through either carboxyl- or amino-terminal peptide extension, so as to promote folding within host cells. Another embodiment relates to a method for enhancing the in vitro renaturation of a protein or polypeptide of interest expressed by recombinant DNA techniques, in circumstances where, following expression, a substantial percentage of the expressed protein or polypeptide of interest is localized within inclusion bodies. Yet another embodiment of the present invention relates to an expression vector comprising a nucleic acid sequence encoding a peptide extension and a multiple cloning site for inserting, in-frame with the peptide extension, a nucleic acid sequence encoding a protein or polypeptide of interest. The peptide extensions of the present invention comprise different amino acid sequences and intrinsic net charges, depending upon the specific species. The total length of the peptide extensions comprise 61 amino acid residues or less, whereas the net intrinsic charges of the peptide extensions range from about −20 to about −2 and from about −20 to about +2, for peptide extensions fused to carboxyl- and amino-termini, respectively. Primary objectives of the present invention include: (i) enhancing the solubility, while concomitantly optimizing the folding, of proteins of interest into their biologically-active conformations in host cells ; (ii) characterizing the features of the carboxyl- and amino-terminal peptide extension that are necessary for their protein folding activity within host cells; (iii) determining whether these carboxyl- and amino-terminal peptide extensions can promote renaturation of mis-folded proteins in vitro; and (iv) identifying protein characteristics which determine behavior of the protein as a substrate for the peptide extension-mediated folding described herein.

This invention was made with Government support under contract number DE-AC02-98CH10886, awarded by the U.S. Department of Energy. The Government has certain rights in the invention.

### FIELD OF THE INVENTION

The present invention relates to an improved method for the production of large quantities of proteins using recombinant genetic-engineering techniques. More specifically, the present invention relates to novel compositions and methods whereby a protein amino- or carboxyl-terminus is modified by fusion to particular peptide sequences which promote folding of the protein into soluble conformations within host cells or during protein refolding in vitro.

### BACKGROUND OF THE INVENTION

Large quantities of biologically active proteins are required for basic studies of protein structure-function relationships and also for the use of proteins in medical or industrial applications. Recombinant DNA technology enables the expression of proteins to unusually high levels in various cell types. In bacteria, protein overexpression is typically accomplished by cloning a DNA fragment encoding the desired protein into a suitable plasmid-based expression vector. Expression vectors contain regulatory sequences which provide for vigorous transcription of the cloned DNA fragment and translation of the corresponding mRNA into the desired protein. Further increases in expression result from the fact that plasmid vehicles replicate to high copy number in bacterial cells, thus providing multiple copies of the expression construct in each transformed cell. Expression vectors also generally include one or more selectable markers (e.g., antibiotic resistance factor), so that the cells which have been successfully transformed with the expression vector can be identified and separated from those cells which have not been transformed.

One of the most powerful prokaryotic systems, with respect to the amounts of the protein of interest that are produced, is the bacteriophage T7-based expression system (Moffatt, B. A. and Studier, F. W. J. Mol. Biol. 189:113-130 (1986)). In this system, the gene or cDNA of interest is cloned downstream of a promoter element derived from the bacteriophage T7 DNA genome. When plasmid DNA containing this recombinant promoter-gene construct is transformed into E. coli strains which also contain the bacteriophage T7 RNA polymerase, the T7 RNA polymerase specifically recognizes the T7 promoter element and generates extraordinary amounts of the corresponding mRNA transcript, leading to overexpression of the recombinant protein within the E. coli host cells. In this and other similar bacterial expression systems, recombinant proteins rapidly become the most prevalent protein species in the host cell.

Although many prokaryotic systems for protein over-expression often perform reasonably well, there are many cases when the proteins expressed by these systems are unable to fold into their native, biologically-active conformations. The resulting misfolded proteins either accumulate in cells as insoluble aggregates (inclusion bodies) or are degraded by host cell proteases. More specifically, many cytosolic proteins and almost all membrane-associated proteins derived from eukaryotic organisms are insoluble when overexpressed in bacterial cells. In addition, endogenous host cell proteins also have been observed to misfold and form insoluble aggregates during overexpression in homologous cell systems.

Understanding the mechanisms by which proteins fold into their native conformations in vivo will likely provide insights into why proteins sometimes misfold during overexpression, and what steps might be taken to surmount this problem. Historically, in vivo pathways of protein folding have been technically difficult to study directly because of the complexity of cell systems. Consequently, most of the detailed knowledge of protein folding has been acquired from relatively simpler model systems of protein refolding in vitro. For example, the classic experiments of Anfinsen demonstrated that denaturation of purified bovine ribonuclease (with urea and reducing agent) results in loss of enzymatic activity, but that enzymatic activity is regained upon removal of the denaturing agents by step-wise dialysis (reviewed in Anfinsen, C. B. (1973) Science 181:223-230). These studies led to the fundamental concept that the amino acid sequence of a protein is necessary and sufficient to specify its biologically-active tertiary conformation, and that trans-acting factors are not required to guide the protein folding process.

Following the lead of Anfinsen, many investigators have attempted to recover recombinant proteins of interest from insoluble aggregates (inclusion bodies). Typically, protein aggregates are dissolved in a concentrated solution of a denaturing agent and then renatured by decreasing the denaturant concentration either rapidly by dilution or slowly by step-wise dialysis. Biologically active proteins have been obtained by application of this method, but in general, single domain proteins can be refolded with better yields than larger multi-domain proteins. Other factors can interfere with in vitro refolding, such as the presence of multiple cysteine residues which may oxidize to form non-native intramolecular or intermolecular disulfide bonds. Re-aggregation of the protein upon removal of the denaturing agent also is a common, unproductive side reaction in these experiments. The tendency to form aggregates increases with protein concentration, therefore refolding experiments must be performed at low protein concentration. At the end of the process, one typically obtains a dilute solution of a heterogeneous mixture of properly and aberrantly folded products. The products must be concentrated and then fractionated chromatographically to separate the properly folded material from other side products of the refolding reaction. Because of the technical difficulties associated with in vitro refolding, most investigators prefer to work with in vivo expression systems if they are available for the protein of interest.

Unlike in vitro protein refolding reactions, nascent proteins fold properly in vivo at very high intracellular protein concentrations. This difference has led many investigators to search for intracellular factors that keep nascent polypeptide chains from aggregating during their synthesis on ribosomes and that promote subsequent folding of the polypeptide chains after their release from ribosomes. Indeed, a class of protein factors known as molecular chaperones has been identified which assists in the folding of many, but not all, nascent proteins in prokaryotic and eukaryotic cells, and in the repair of protein conformational damage incurred during environmental stress (see: Frydman, J. (2001) Annu. Rev. Biochem. 70:603-47). Prokaryotic cells contain several different protein species which have chaperone-like activity (e.g. DNA-K, Trigger factor, GroEL, etc.). These chaperones are present constitutively in normal cells at low concentrations, and may be synthesized at elevated levels following heat shock, nutrient starvation, or other stressful conditions that can damage the conformation of mature proteins or interfere with the synthesis of nascent proteins in vivo. Chaperones have been shown to play important roles in the repair of protein conformational damage resulting from environmental stress and in the degradation of proteins that are damaged beyond repair. However, it is clear that chaperones also assist in the folding of many nascent polypeptides under normal conditions. If cells are unable to synthesize normal levels of chaperones, as is likely the case during protein overexpression when most of the cell's translation machinery is re-directed towards the production of the overexpressed recombinant protein species, then the resulting deficit in chaperone activity might result in the misfolding of overexpressed protein species whose proper folding under normal conditions requires the assistance of chaperones. Several investigators have attempted to rescue folding of overexpressed proteins by co-expression of chaperones, but this approach has resulted at best in only modest increases in the yield of properly folded protein.

The expression of the protein or polypeptide of interest as a fusion protein has also been proposed as a method for averting protein misfolding and inclusion body formation (see Snavely, U.S. Pat. No. 6,077,689; Mascarenhas, et al. U.S. Pat. No. 5,563,046; and Harrison, et al., U.S. Pat. Nos. 5,989,868 and 6,207,420). Considerable effort has also been devoted to the development of various fusion partners to either protect the protein or polypeptide of interest from degradation by host cell proteases or to provide a facile means of purification of the protein or polypeptide of interest (reviewed by Ford, et al., (1991) Prot. Exp. and Purif. 2:95-107). It has been suggested that such fusion elements may also serve to enhance the solubility of the recombinant protein of interest. Drawbacks of such fusion systems for enhancing the solubility of recombinant proteins in the host cells include the fact that the applicability of each system to a wide variety of proteins of interest is not known, the fusion partners tend to be large polypeptides thus decreasing the relative yield of the protein or polypeptide of interest, and for the most part such systems include the necessity of engineering a specific cleavage site into the fusion protein so that the protein or polypeptide of interest can be separated from its fusion partner, which many times requires the use of costly reagents to effect that cleavage. Furthermore, the expression of proteins and polypeptides of interest as fusion proteins does not always avert the formation of inclusion bodies.

Accordingly, there remains an as yet unfulfilled need for the development of expression methodologies to ameliorate problems associated with solubility and folding during the overexpression of both foreign and endogenous proteins in high-yielding protein expression systems.

### SUMMARY OF THE INVENTION

The present invention relates to novel compositions and methods whereby a protein or polypeptide of interest is modified, through the use of recombinant DNA technology, by extending the protein carboxyl- or amino-terminus with peptides which promote folding of the protein within host cells (e.g., prokaryotic cells such as E. coli or eukaryotic cells such as yeast, insect and mammalian cells) or refolding of the protein in vitro. For example, the present invention relates to methods and compositions that may be utilized to express proteins or polypeptides which are insoluble and/or are incapable of adopting a biologically active (i.e., native) conformation if expressed without the peptide extension of the present invention. Additionally, in cases when fusion of the protein of interest to the peptide extensions of the present invention does not directly yield a soluble protein folded into a native conformation in vivo, the present invention relates to methods and compositions which may facilitate in vitro refolding of fusion proteins obtained from insoluble protein aggregates (e.g. inclusion bodies). Thus, the methods and compositions disclosed herein have provided markedly-improved results over those discussed in the prior art, with a significant fraction of proteins in a test set recovered, at least in part, as soluble products folded into their native, biologically-active conformation.

Primary objectives of the present invention include: (i) enhancing the solubility, while concomitantly optimizing the folding, of proteins of interest into their biologically-active conformations in host cells ; (ii) characterizing the features of the carboxyl- and amino-terminal peptide extension that are necessary for their protein folding activity within host cells; (iii) determining whether these carboxyl- and amino-terminal peptide extensions can promote renaturation of mis-folded proteins in vitro; and (iv) identifying protein characteristics which determine behavior of the protein as a substrate for the peptide extension-mediated folding described herein.

In one embodiment, the present invention relates to a method for enhancing the solubility and promoting the adoption of the native folded conformation of a protein or polypeptide expressed by recombinant DNA techniques in a cell, which involves providing a first nucleic acid sequence encoding a protein or polypeptide of interest and a second nucleic acid sequence encoding a peptide extension which is 61 amino acid residues or less in length. The nucleic acid encoding the peptide extension (i.e., the second nucleic acid) and the nucleic acid encoding the protein or polypeptide of interest (i.e., the first nucleic acid) are fused such that the encoded peptide extension is fused either to the carboxyl- or amino-terminus of the protein or polypeptide of interest. Specifically, the nucleic acid encoding the peptide extension, the nucleic acid encoding the protein or polypeptide of interest, and an appropriate expression vector are fused and transformed into a host cell, thus allowing expression of a fusion protein comprising the protein or polypeptide of interest and the peptide extension. The expressed fusion protein comprises a properly folded protein or polypeptide of interest and a peptide extension of the present invention having a non-ordered (i.e., random) conformation.

In another embodiment, the present invention relates to a method for enhancing the in vitro refolding of a protein or polypeptide expressed by recombinant DNA techniques in a cell. This method applies specifically to the expression of a protein or polypeptide of interest in a cell where a substantial percentage of the expressed protein or polypeptide is localized within macroscopic inclusion bodies. The method involves providing a first nucleic acid sequence encoding a protein or polypeptide of interest and a second nucleic acid sequence encoding a peptide extension which is 61 amino acid residues or less in length. The nucleic acid encoding the peptide extension (i.e., the second nucleic acid sequence) and the nucleic acid encoding the protein or polypeptide of interest (i.e., the first nucleic acid sequence) are fused such that the encoded peptide extension is fused to either the carboxyl- or amino-terminus of the protein or polypeptide of interest. Specifically, the nucleic acid encoding the peptide extension, the nucleic acid encoding the protein or polypeptide of interest, and an appropriate expression vector are fused and transformed into a host cell, thus allowing expression of a fusion protein comprising the protein or polypeptide of interest and the peptide extension. Following expression, the inclusion bodies are isolated from lysates of the cell and treated with a denaturing solution (e.g., guanidine hydrochloride or urea) so as to denature and solubilize the fusion proteins comprising the inclusion bodies. The denatured proteins are then suspended in a renaturation buffer by dilution or dialysis in order to allow the fusion protein to obtain its native conformation and solubility. In the renaturation process the protein or polypeptide of interest adopts its native, biologically active conformation while the peptide extensions of the present invention adopt a non-ordered (i.e., random) conformation.

Yet another embodiment of the present invention relates to expression vectors comprising a nucleic acid sequence encoding a peptide extension of the type described above, and a multiple cloning site for inserting, in-frame with the nucleic acid encoding the peptide extension, a nucleic acid sequence encoding a protein or polypeptide of interest.

### BRIEF DESCRIPTION OF THE FIGURES

FIG. 1: Illustrates the schematic organization and expression constructs of the coxsackievirus and adenovirus receptor (CAR).

Panel A: Illustrates a schematic of the CAR structural organization. The amino-terminal signal peptide is represented as a shaded box. The extracellular region of CAR consists of two structural domains (D1 and D2), each of which have β-sandwich-type folds similar to those of immunoglobulin domains (hence CAR is categorized as a member of the “immunoglobulin superfamily” of proteins). The single hydrophobic membrane-spanning region and the intracellular region are indicated (i.e., TM and CYT, respectively).

Panel B: Illustrates the nucleic acid sequences of the forward and reverse PCR primers used to amplify CAR D1 (the complement of the reverse primer sequence is shown). Both primers were tailed with restriction sites (bold type) to facilitate cloning into the pET15b expression vector. Amino acid residues encoded by the primers are shown in single letter code.

Panel C: Illustrates the nucleotide and amino acid sequences of the CAR D1-T7A fusion protein generated by ligation of the CAR D1 PCR product (shown in panel B) to the pET15b expression vector (both the PCR product and the pET15b plasmid were digested with NcoI and XhoI before ligation). The amino acid sequence of the resulting CAR D1-T7A fusion protein is shown in single letter code on the top line (note that the central amino acid residues of CAR D1, from Ile 3 to Ala 125, are not shown, and are represented by . . . ). The translation termination signal is indicated by *. Nucleotide sequences of restriction enzyme cleavage sites used to generate CAR D1-peptide fusion proteins are labeled and shown in bold type.

FIG. 2: Illustrates the integrated net charge of CAR D1 and A33 D1 polypeptides plotted against polypeptide fractional length. Running tally of polypeptide net charge (calculation based only upon D, E, R, and K residues) was plotted as a function of polypeptide fractional length (i.e., charged residue number/total length of polypeptide×100). Solid line, A33 D1; Dotted line, CAR D1; Horizontal dotted line, position of uncharged species.

FIG. 3: Illustrates a schematic of the structure of vectors for fusion of a protein amino-terminus to peptide extensions. DNA fragments encoding the T7B peptide or various modified T7B peptides were amplified by PCR using primers that appended an upstream NcoI restriction site and a downstream NdeI restriction site, as shown in Panel A. The PCR products were then cloned between the NcoI and NdeI sites of pET15b, as shown in Panel B. In the final ligated products, the 6-His tag (which is normally present in pET15b) is replaced by the N-terminal peptides.

### DETAILED DESCRIPTION OF THE INVENTION

### Definitions

The following definitions are provided to assist in providing a clear and consistent understanding of the scope and detail of the terms utilized herein.

Amino Acids: Amino acids are shown either by one letter or three letter abbreviations as follows: A Ala (Alanine); C Cys (Cysteine); D Asp (Aspartic acid); E Glu (Glutamic acid); F Phe (Phenylalanine); G Gly Glycine; H His (Histidine); I Ile Isoleucine; K Lys (Lysine); L Leu (Leucine); M Met (Methionine); N Asn (Asparagine); P Pro (Proline); Q Gln (Glutamine); R Arg (Arginine); S Ser (Serine); T Thr (Threonine); V Val (Valine); W Trp (Tryptophan); Y Tyr (Tyrosine).

Biologically Active: In reference to proteins, biological activity is the function normally performed by said protein in a biological system, and biologically active refers to the capacity of a protein to carry out its normal function in a biological system and also in vitro.

Cloning Vector: A plasmid DNA, phage DNA, cosmid, or other DNA sequence that is able to replicate within a host cell, which is characterized by one or more restriction endonuclease recognition sites at which such DNA sequences may be cut in a determinable fashion without attendant loss of an essential biological function of the DNA (e.g. replication, production of coat proteins or loss of promoter or binding sites), and which contain a marker suitable for use in the identification of transformed cells (e.g., antibiotic resistance, bacterial colony color selection or auxotrophy complementation). A cloning vector is often called a vehicle.

Conformation: As utilized herein, the term “conformation” is defined as the three-dimensional arrangement of amino acid residues in a polypeptide or protein. Amino acid sequence dictates the conformation of the polypeptide or protein, whereas the conformation imparts biological activity. The term “native conformation” is defined as the three-dimensional arrangement of the polypeptide or protein in vivo or under physiological conditions in vitro, and is typically the three-dimensional arrangement which is required for biological activity.

Expression: The process by which a polypeptide is produced from a gene or DNA sequence. It is a combination of transcription and translation. Recombinant protein expression refers to the in vivo synthesis of a desired protein using an expression vector. Overexpression refers to production of a desired protein in amounts such that the expressed protein becomes the most prevalent protein species in the host cell.

Expression Vector: As utilized herein, the term “Expression Vector” is comprised of all the elements (e.g., vector, promoters, termination sequences, and the like) which are required for the in vivo transcription and subsequent translation of a protein of interest by a host cell. An expression vector construct is an expression vector in which the nucleic acid encoding a desired protein has been inserted in such a way that the protein will be expressed following introduction of the construct into an appropriate host cell coupled with appropriate culturing conditions. Although the use of prokaryotic expression vectors is expressly taught herein, the present invention may also utilize eukaryotic expression vectors for the expression of a protein of interest. Vectors may be specific, or optimized, for use in prokaryotic or eukaryotic cells. Features which render an expression vector specific for use in a prokaryotic or eukaryotic cell are well known by those skilled in the art.

Folding: As utilized herein, the term “folding” refers to the process by which an amino acid sequence, polypeptide or protein acquires its three-dimensional structure (conformation). Folding is utilized herein to refer to the acquisition of a native conformation by the amino acid sequence, polypeptide or protein, whereas the term mis-folding or mis-folded refers to the acquisition of a non-native, non-biologically active conformation by an amino acid sequence, polypeptide or protein.

Fusion Protein: As utilized herein, the term “fusion protein” refers to a chimeric protein or polypeptide comprising a protein or polypeptide of interest and an unrelated protein, polypeptide or peptide extension. As utilized herein, a fusion protein is one which is produced by expression from an expression vector construct of a nucleic acid sequence encoding the protein, polypeptide or peptide extension in frame with the sequence of the polypeptide or protein of interest.

Inclusion Body(ies): As utilized herein, the term “inclusion body(ies)” refers to aggregates of insoluble protein which are formed during over-expression of some proteins or polypeptides in bacterial and other host cells.

### DESCRIPTION OF THE INVENTION

The present invention relates to novel compositions and methods whereby a protein of interest (e.g., a recombinant protein) is modified through either carboxyl- or amino-terminal peptide extension, so as to promote folding or enhance solubility within a host cell (e.g., prokaryotic cells such as Escherichia coli, or eukaryotic host cells including yeast, insect and mammalian cells). For example, one embodiment of the present invention relates to methods and compositions that may be utilized to express proteins or polypeptides which are insoluble and/or are incapable of adopting a biologically active (i.e., native) conformation if expressed without the peptide extension of the present invention. Another embodiment relates to methods and compositions which facilitate the in vitro refolding of denatured fusion proteins in cases where fusion of the protein carboxyl- or amino terminus to a peptide extension does not directly yield a soluble protein or polypeptide folded into a native conformation in vivo. Thus, the methods and compositions disclosed herein have provided markedly improved results over those discussed in the prior art.

A primary objective of the present invention is to enhance the solubility, while concomitantly optimizing the folding of recombinant proteins of interest into their biologically active conformations in host cell organisms. Accordingly, the methodologies disclosed in the present invention will enable the production of biologically-active proteins derived from mammalian, other non-prokaryotic sources and from prokaryotic sources within host cells in quantities sufficient for biochemical and biophysical analyses, such as X-ray crystallography. The novel technical advances disclosed herein also further the general understanding of the basic mechanisms of protein folding in vivo. As previously discussed, the folding of many proteins in vivo is believed to be assisted by “chaperones”, whereby the absence or insufficiency of appropriate chaperones during over-expression of recombinant proteins may account for the misfolding and aggregation of many recombinant proteins which are expressed in host cells. As discussed supra, previous attempts at solving this problem have typically involved the modification of the host bacterial cells to co-express appropriate chaperones. Uniformly, these aforementioned attempts have met with limited success.

Other primary objectives of the present invention included: (i) characterizing the features of the carboxyl- and amino-terminal peptide extension that are necessary for their protein folding activity within prokaryotic cells; (ii) determining whether these carboxyl- and amino-terminal peptide extensions can promote renaturation of mis-folded proteins in vitro; and (iii) identifying protein characteristics that determine the behavior of proteins as substrates for peptide extension-mediated folding in vivo.

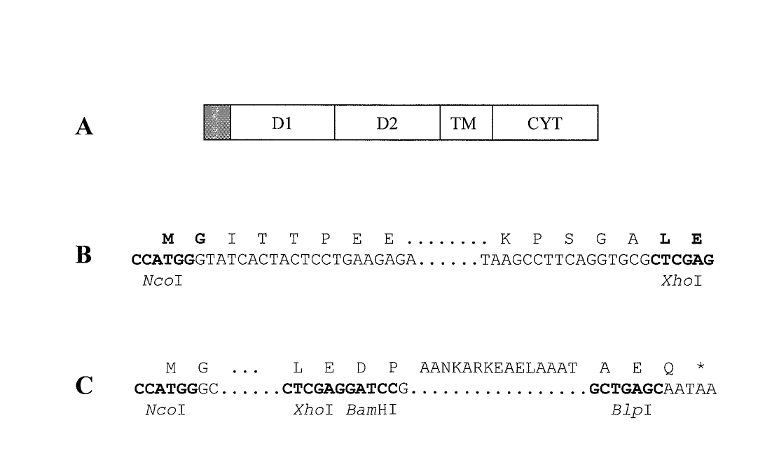

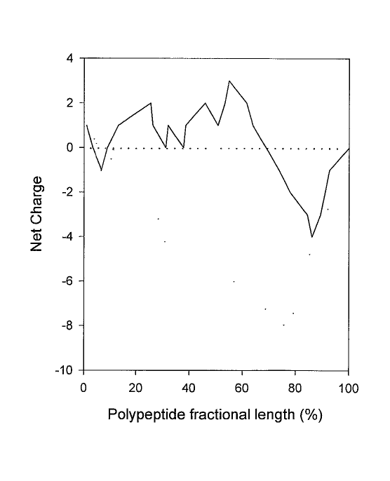

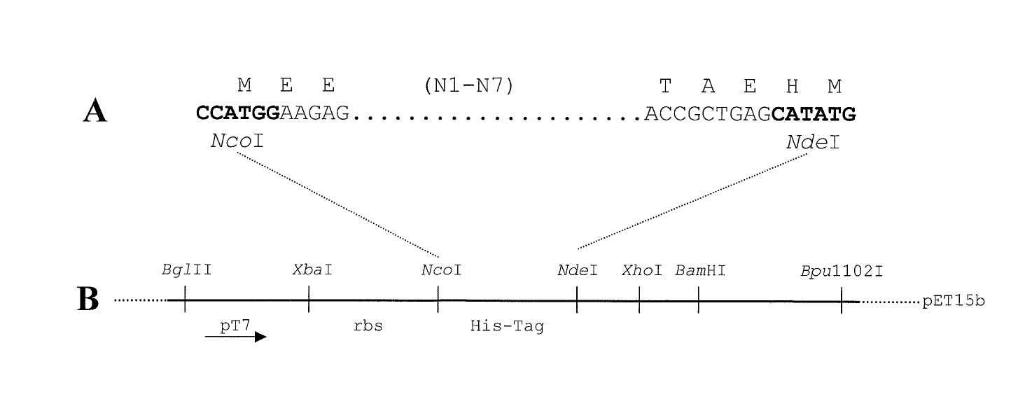

In brief, the present invention relates to compositions and methods relating to fusion of the carboxyl- or amino-terminus of proteins of interest to peptides which increase protein solubility or alter protein folding pathways, thereby promoting the folding of proteins into their correct, biologically active conformations. In one example, the nucleic acid encoding a peptide extension and the nucleic acid encoding the extracellular domain (D1) of the human membrane receptor for coxsackievirus and adenovirus (CAR) were fused such that the encoded peptide extension was fused to the carboxyl-terminus of encoded CAR D1 (see FIG. 1). It should be noted that unmodified (i.e., non-peptide-extended) CAR D1 mis-folds and forms insoluble inclusion bodies when expressed in E. coli Augmentation of CAR D1 folding was found to be sequence- and intrinsic net charge-specific, with respect to carboxyl-terminal extensions, as folding of the CAR protein was not rescued by fusion to other peptides of similar length, but different sequence and intrinsic net charge.

Thus, in one embodiment, the present invention relates to a method for enhancing the solubility of, and promoting the adoption of native folding conformation, of a protein or polypeptide expressed by recombinant DNA techniques in a host cell. A first nucleic acid sequence is provided, which encodes the protein or polypeptide of interest. In connection with this invention, the protein or polypeptide of interest is one which is substantially insoluble or biologically inactive, when expressed in the host cell by recombinant DNA techniques.

A second nucleic acid sequence is provided which encodes a peptide extension having a net negative charge. The second nucleic acid is fused in-frame to the first nucleic acid in an expression vector such that a fusion protein encoded by the first and second nucleic sequences is expressed in the host cell following transformation of the host cell with the expression vector encoding the fusion protein. The peptide extension encoded by the second nucleic acid sequences is positioned at the carboxyl-terminus of the protein or peptide of interest. The peptide T7A of Table 1 is specifically excluded in connection with this embodiment. In any jurisdiction which does not recognize a one-year grace period for filing a patent application following the public disclosure of an invention, it may also be necessary to exclude peptides T7B and T7C of Table 1 in connection with this and related embodiments.

A host cell is then transformed with the expression construct described above, and the transformed host cell is cultured under conditions appropriate for the expression of the fusion protein. As demonstrated in the Exemplification section which follows, prokaryotic cells (e.g., E. coli), represent an important example of a host cell to which the invention applies. However, solubility problems and the formation of inclusion bodies are well-known in eukaryotic host cells as well. The fundamental principles of the present invention apply with equal force in a eukaryotic host cell background.

In general, the present invention relates to two types of fusions. In a first type, the protein or polypeptide extension is attached at the carboxyl-terminus of the protein or polypeptide of interest. In the second type, the protein or polypeptide extension is attached at the amino-terminus of the protein or polypeptide of interest.

In connection with the first type of fusion, the peptide extension carries a net negative charge which ranges from about −2 to about −20. The effect of the charged extension on the solubility or biological activity of the protein or polypeptide of interest can vary depending upon the magnitude of the net negative charge. Therefore, preferred ranges of from −2 to −4; from −5 to −9; from −10 to −14; and from −15 to −20 have been specifically described.

Experimental work has revealed no specific extension peptide conformation or structural feature other than net negative charge which is required for the desired activity. The largest of the peptide extensions which have been employed to date is 61 amino acid residues in length. One of skill in the art would recognize that this does not represent an upper theoretical or practical limit on the size of the useful extension.

While not wishing to be bound by theory, it is thought that the strong repulsive force associated with the net negative charge of the peptide extension serves to segregate individual protein or polypeptide molecules following their release from the ribosome. This repulsion serves to provide enough time for the protein or polypeptide to assume their native conformation even at high protein concentration. In the absence of repulsive extension, the proteins tend to aggregate during the folding process forming insoluble inclusion bodies.

A number of peptide extensions representing variations of the 57 residue carboxyl-terminal portion of the T7 10B protein are exemplified below. The present invention encompasses peptide extensions that include this 57 residue polypeptide, or portions thereof, which retain the ability to enhance solubility or biological activity of a protein or polypeptide of interest when expressed as a fusion partner. Also disclosed below are variants of the 57 residue polypeptide (or portions thereof) in which amino acid substitutions were made that maintained the overall net negative charge of between −2 and −20. Such variants are also included within the scope of the present invention.

Examples of specific peptide extensions falling within the scope of the present invention include peptides T7C, T7B, T7B1, T7B2, T7B3, T7B5, T7B6, T7B7, T7B8, T7B9, T7B10, T7B11, T7B12, T7B13, T7A1, T7A2, T7A3, T7A4 and T7A5, as shown in Table 1.

The above relates primarily to fusions in which the peptide extension is attached to the carboxyl-terminal residue of the protein or polypeptide of interest. The present invention also relates to fusions in which the peptide extension is attached to the amino-terminal residue of the protein or polypeptide of interest. This embodiment also applies to expression in prokaryotic and eukaryotic cells. As demonstrated in the exemplification section which follows, the charge range identified to be useful in connection with this embodiment is from about +2 to about −20. As was discussed with carboxyl-terminal peptide extensions, the degree of solubility or activity enhancement varied depending upon the magnitude of the peptide extension charge. Therefore, preferred ranges of from −15 to −20; from −10 to −14; from −5 to −9; from −1 to −4; and from +2 to −1 are specified. No critical structural features of the peptide extension have been observed.

The specific amino-terminal peptide extensions exemplified comprise solubility or activity promoting portions of the 57 residue carboxyl-terminal portion of the T7 gene 10B protein, or variants thereof which result in the maintenance of a net charge ranging +2 to −20. Specifically, disclosed peptides include the following peptides which appear in Table 1: peptides N1, N2, N3, N4, N5, N6 and N7.

In addition to the methods discussed above, the present invention also relates to methods for enhancing the in vitro renaturation of a protein or polypeptide expressed by recombinant DNA techniques in a host cell. This aspect of the invention relates to a protein or polypeptide of interest, a substantial percentage of which is localized in inclusion bodies following expression in the host cell. Like other embodiments of the present invention, the host cell can be prokaryotic or eukaryotic. This embodiment also includes the construction of a recombinant fusion protein having either a carboxyl-terminal or an amino-terminal peptide extension. The nature of the peptide extension of the present embodiment is identical to the nature of the peptide extensions of previously described embodiments.

Following expression of the fusion protein in the host cell, inclusion bodies are isolated from lysates of the host cell. The isolated inclusion bodies are then contacted with a denaturing solution thereby denaturing the fusion protein. Solutions of urea or guanidine hydrochloride are examples of appropriate denaturing solutions. The fusion protein comprising the inclusion bodies is solubilized in a denatured form by the denaturing solution. The fusion protein is then suspended in a renaturation buffer (e.g., a buffered saline solution) by dilution or dialysis in order to allow the fusion protein to obtain its native conformation and solubility. Quantitative recovery of soluble, peptide-extended product, was observed (see Exemplification section which follows).

It was observed (e.g., by HPLC analysis and non-denaturing gel electrophoresis) that a large percentage of this completely soluble fraction was present in solution as aggregate material. Surprisingly, the treatment of this soluble aggregate material by a subsequent heat denaturation step resulted in substantial disaggregation. This was observed even when working with extremely high concentrations of peptide extended protein (e.g., 1 mg/ml or higher).

The present invention also relates to expression vectors which carry sequences encoding peptide extensions of the type described above. The expression vectors are specific for, or optimized for, use with prokaryotic or eukaryotic cells. The features of such vectors which render them specific for a prokaryotic or eukaryotic cell type are well known to those skilled in the art. These features include, without limitation, replicons, transcription signals, termination signals, and the like. The vectors also contain a multiple cloning site which facilitates the insertion of DNA encoding a protein or polypeptide of interest, in-frame with the sequence encoding the peptide extension. The position of the multiple cloning sites relative to the sequence encoding the peptide extension can be oriented such that the peptide extension is attached to the protein or polypeptide of interest at its amino terminus or carboxy terminus.

In another aspect, the present invention relates to antibodies (either monoclonal or polyclonal) which bind specifically to the peptide extensions of the present invention. Such antibodies are useful, for example, in the isolation of a fusion protein comprising such a peptide extension. Methods of making such antibodies are well known in the art. In preferred embodiments, the antibodies of the present invention are characterized by the ability to specifically bind one or more peptide extensions from the set described in Table 1.

There are several possible mechanisms for the carboxyl-terminal peptide extension-enhanced solubility and folding of the over-expressed proteins of the present invention (e.g., CAR D1). While not wishing to be bound by any single theory, one possible mechanism is that the strong repulsive force between highly-charged peptide extensions blocks aggregation of proteins that have non-native conformations during translation (for N-terminal peptide extensions) or after release of the nascent polypeptide from ribosomes (for both N- and C-terminal peptide extensions). The blocking of aggregation provides time for the solvent-exposed, nascent polypeptide chains to proceed along the folding pathway, both during translation and after release from the ribosome, and ultimately to adopt the native folded conformation. In such a mechanism, the highly charged peptide extensions may compensate for deficits in chaperone activity that result from over-expression of the protein or polypeptide of interest encoded by the expression vector. Thus, the fused peptide extension and the protein or polypeptide of interest may represent a self-chaperoning system which facilitates its own solubility and proper folding of the protein or polypeptide of interest. In vitro protein refolding may be enhanced by a similar mechanism.

The possible mechanism(s) for amino-terminal peptide extension-mediated folding may be more complex than that of carboxyl-terminal extensions. While not wishing to be bound by any single theory, in some cases (e.g., CAR D1) the amino-terminal peptides may circumvent nascent chain precipitation by changing the net charge on the nascent polypeptide chain early on in the course of its synthesis, thus acting through a similar electrostatic repulsion mechanism as the carboxyl-terminal peptides extensions. However, in cases when the peptide extension itself has only a small net charge or no net charge, the rescue of protein folding must occur through an alternate mechanism unrelated to electrostatic charge repulsion. One explanation might be that because amino-terminal peptide extensions are present on the growing nascent polypeptide chain from the onset of translation, they may cause the nascent polypeptides or proteins of interest to go through a novel set of folding intermediates in which the tendency to form aggregates is diminished.

### Exemplification

Specific exemplifications of the compositions and methods of the present invention will now be fully discussed below.

I—Generation of the pET15b-CAR D1 Construct

The cellular receptor for adenovirus type 2 (Ad2), and many other adenovirus serotypes, has been recently described. The receptor, encoded by a single gene on human chromosome 21, also serves as the cellular receptor for group B coxsackieviruses (CBV; Tomko, et al., Proc. Natl. Acad. Sci. U.S.A. 94: 3352-3356 (1997)). Accordingly, this receptor was designated the coxsackievirus and adenovirus receptor (CAR). CAR is a 46 kiloDalton (kDa) member of the immunoglobulin-superfamily (IgSF) that possesses an extracellular aspect comprising of an amino-terminal domain (D1) which has a protein fold related to that of immunoglobulin (Ig) variable domains, and an adjacent domain (D2) whose fold is related to that of Ig constant region domains. CAR has a single, hydrophobic, membrane-spanning region and a ˜100 residue cytoplasmic domain. See, Bergelson, et al, Science 275: 1320-1323 (1997); Tomko, et al., (1997). The structural organization of the CAR domains is illustrated schematically in FIG. 1, Panel A.

The pET15b vector (Novagen) was derived in part from the bacteriophage T7 gene 10 transcription unit and includes a DNA fragment which contains both the transcription terminator and the last 18 codons (codons 381-398) of the gene 10B protein structural gene. See, Studier, et al., Methods Enzymol. 185: 60-89 (1990).

In the present invention, a complementary DNA (cDNA) fragment encoding the CAR D1 domain was amplified by polymerase chain reaction (PCR) and cloned into the NcoI and XhoI sites of expression vector pET15b (see, Freimuth, et al., J. Virol. 73: 1392-1398 (1999)). The resulting construct was designated pET15b-CAR D1.

More specifically, a cDNA fragment encoding the human CAR D1 domain was obtained by reverse-transcription PCR (RT-PCR) amplification of total RNA from murine A9 cells that were transfected with the cloned human CAR gene. The nucleotide sequence of the CAR D1 -encoding cDNA fragment corresponded exactly to the CAR cDNA sequence reported in GenBank file Y07593. First-strand cDNA synthesis was primed with oligo(dT). Forward and reverse PCR primers were then used to amplify the cDNA fragment encoding CAR D1. Sequences of the PCR primers used are shown in FIG. 1, Panel B. Restriction sites for NcoI and XhoI (shown in bold type) were incorporated into the forward and reverse PCR primers to facilitate cloning into the pET15b expression vector. Following digestion with the restriction endonucleases NcoI and XhoI, the cDNA fragment encoding CAR D1 was cloned into the NcoI and XhoI sites of expression vector pET15b. The resulting construct was designated pET15b-CAR D1.

In the pET15b-CAR D1 construct, the 3′-terminus of the CAR D1 cDNA fragment and the last 18 codons of gene 10B were joined in-frame to create a fusion protein in which the carboxyl-terminus of CAR D1 was extended with a 22 residue peptide (T7A peptide, see Table 1). The pET15b-CAR D1 construct is illustrated in FIG. 1, Panel C.

II. Expression of the CAR D1-T7A Fusion Protein

Expression of the CAR D1 -T7A fusion protein (the sequence of the T7A peptide extension is shown in Table 1) from the pET15b-CAR D1 construct was performed as follows. The pET15b-CAR D1 construct was transformed into Escherichia coli strain BL21-DE3 (Novagen, Inc.). Freshly transformed colonies were used to inoculate Luria-Bertani (LB) broth containing 150 mg/L penicillin G (Sigma), and the culture was grown at 37° C. until mid-log phase (optical density approximately 0.8 at 600 nm). The culture was then chilled to 18° C. and adjusted to 50 μM isopropyl D-thiogalactopyranoside (IPTG; Aldrich-Sigma) to induce protein expression. After incubation for an additional 5-20 hr at 18-20° C., the cells were harvested and analyzed for expression of CAR D1. Cells were lysed by several cycles of rapid freezing and thawing in the presence of lysozyme, followed by sonic disruption with a probe tip sonicator (Heat Systems, Inc.). Lysates were then centrifuged, and the supernatant fraction was transferred to a fresh tube. Protein content in both the soluble (supernatant) and insoluble (pellet) fractions was examined by SDS-PAGE (electrophoresis in polyacrylamide gels in the presence of sodium dodecylsulfate, a strong detergent and protein denaturant). Experimental results demonstrated that, when CAR D1 was fused to the 22 residue T7A peptide extension, approximately 50% of the CAR D1 protein was present in the soluble fraction of cell lysates, whereas the remainder of the CAR D1 fusion protein was present in the insoluble pellet fraction (which contained the macroscopic inclusion bodies). In contrast, when the 22 residue peptide extension was eliminated by insertion of a stop codon upstream of the XhoI cloning site, the CAR D1 fusion protein was found to be completely aggregated into insoluble inclusion bodies (See, Freimuth, et al. (1999)).

CAR D1 -T7A fusion protein was purified from the soluble fraction of induced cell lysates by precipitation with ammonium sulfate (35 to 60% cut at 25° C.) followed by anion-exchange chromatography (on DE52, Whatman) in 10 mM Tris-HCl buffer (pH 7.5). Approximately 5 mg of partially-purified CAR D1-T7A fusion protein was recovered from 1 liter of culture. It was further demonstrated that the peptide extension could be removed from the soluble, purified CAR D1 -T7A fusion protein by limited proteolytic-digestion with trypsin (having a major site of action at arginine and lysine residues), and that the resultant trypsin-stable CAR D1 fragment remained in solution and was biologically-active. See, Bewley, et al., Science 286: 1579-1583 (1999). Thus, the results obtained in these initial studies demonstrated that the bacteriophage T7-derived T7A peptide extension mediated the folding of CAR D1 into its biologically active conformation in E. coli.

III. Specificity of the Peptide-Mediated Folding of CAR D1

Additional experiments were performed in order to establish whether the mechanism of CAR D1 folding enhancement was specific for the T7A peptide derived from the bacteriophage T7 gene 10B protein. The bacteriophage T7 gene 10 encodes two proteins, 10A and 10B, which are identical in amino acid sequence for the first 342 amino acid residues. Translation of the 10A protein is continued for three additional codons before terminating after codon 345, whereas a reading frame shift in codon 343 produces the 10B form which continues translation for a total of 56 additional codons before terminating after codon 398. See, Condron, et al., J. Bacteriol. 173: 6998-7003 (1991). The sequence of the carboxyl-terminal 57 amino acid residues of the bacteriophage T7 gene 10B protein (amino acid residues 343-398) is FQSGVMLGVASTVAASPEEASVTSTEETLTPAQEAARTRAANKARKEAELAAATAEQ. The bacteriophage T7 gene 10A and 10B proteins are structural proteins that form the icosahedral phage head. The unique 57 residue carboxyl-terminus of the 10B protein is exposed on the surface of phage heads, but this peptide is not essential for propagation of bacteriophage T7 under laboratory conditions. Indeed, in the bacteriophage T7-based phage display system (see Novagen catalog and Studier, et al. U.S. Pat. No. 5,766,905), foreign peptides are substituted for the non-essential 10B C-terminal 57 residue peptide, and thus become displayed on the phage head.

Bacteriophage T3 (a close relative of T7) also has two forms of its major capsid protein (these 2 bacteriophage T3 proteins are also named the gene 10A and 10B proteins) that are generated by a similar frameshift event (see, Condreay, et al., J. Mol. Biol. 207: 555-561 (1989)). However, the carboxyl-terminal peptides of the T3 and T7 gene 10B proteins are not conserved in amino acid sequence (see Table 1) or in length (89 residues long in T3 vs 57 residues in T7).

To investigate the specificity of the T7A peptide-mediated folding of CAR D1, the effects of bacteriophage T7 and T3 gene 10B-derived, carboxyl-terminal peptide extensions on the folding of CAR D1 were compared. The DNA fragment encoding the 18 amino acid residue T7A peptide was excised from the pET15b-CAR D1 construct by digestion with restriction endonucleases BamHI and BlpI (see, FIG. 1, Panel C) and replaced with PCR products encoding either: (i) the complete 57 amino acid residue T7 gene 10B terminal peptide (T7C); (ii) a shorter fragment encoding the terminal 40 amino acid residues of the T7 gene 10B terminal peptide (T7B); or (iii) a fragment encoding the terminal 39 amino acid residues of the bacteriophage T3 gene 10B terminal peptide (T3). These peptide extensions were designated Peptide T7C, Peptide T7B, and Peptide T3. The amino acid sequences of these peptide extensions are shown in Table 1.

Electrophoretic results demonstrated that fusion to either of the longer T7-derived peptide extensions (i.e., T7B and T7C) rendered CAR D1 completely soluble, even when protein expression was induced at 37° C. (the T7A peptide was only effective at folding CAR D1 when protein expression was induced at temperatures below 25° C.). In contrast, CAR D1 was completely insoluble when fused to the T3-derived peptide extension. As shown in previous experiments, CAR D1 devoid of any carboxyl-terminal peptide extension was completely insoluble, whereas the CAR D1 protein was only partially soluble when fused to the initial 22 residue T7A peptide extension.

Similarly, as had been demonstrated (Bewley, et al., (1999)) for Peptide T7A, Peptide T7B could be cleaved from the soluble CAR D1 fusion protein by limited proteolysis with trypsin. Furthermore, the resultant trypsin-stable CAR D1 fragment was capable of binding specifically to the adenovirus fiber knob domain and was also recognized by antibodies prepared against CAR D1. In contrast, the CAR D1 -T3 fusion protein isolated from inclusion bodies was completely hydrolyzed by low concentrations of trypsin, thus indicating that the CAR D1 component of this fusion protein was misfolded. Accordingly, the two longer T7-derived peptides, but not the T3-derived peptide, are able to mediate quantitative folding of CAR D1 into its biologically-active conformation in E. coli.

The failure of the T3-derived peptide to mediate CAR D1 folding suggested that the folding of CAR D1 results from some characteristic(s) of the T7 peptides that is not shared by the T3 peptide extension. In support of this view, the T3 and T7 terminal peptides share no obvious sequence homology (see, Table 1). Because fusion of CAR D1 to the two longer T7-derived peptides (T7B and T7C) resulted in 100% solubilization and folding, this analysis also suggests that Peptides T7B and T7C contain a feature(s) or characteristic(s) that is (are) not present or only partially present in the shorter 22 amino acid T7A peptide. Experiments were performed to determine the basis for the complete CAR D1 folding activity of the two longer peptides, as described herein below.

IV. Mechanism of Protein Folding by T7-Derived Peptide Extensions

A. Role of predicted amphipathic α-helices. Both the T7B and T7C peptides were predicted by sequence analysis algorithms (e.g., Chou/Fasman) to contain two long α-helices, both of which have weak amphiphilic character as revealed by helical wheel projections. It is conceivable that peptide extensions with weak amphiphilic character could function as cis-acting chaperones by interacting transiently with hydrophobic regions of the newly translated polypeptide to prevent aggregation. Accordingly, peptide extension mutants were constructed to determine if amphiphilic α-helical character is necessary for the protein folding activity of these peptides. Peptides T7B2 and T7B3 incorporate helix-disrupting proline or glycine residues at the start of the predicted carboxyl-terminal helix, whereas Peptide T7B1 has a deletion that would disrupt the amphiphilic character of the predicted helix. None of these three modified peptide extensions reduced the yield of soluble CAR D1 produced in E. coli. Thus, these results demonstrate that the folding activity of the T7B and T7C peptide extensions does not depend on the ability of these peptides to form amphiphilic α-helices.

B. Recruitment of trans-acting chaperones. Experiments were then performed to test whether the T7B peptide functions by recruiting chaperones to the nascent fusion protein, thus enhancing its folding. The ClpB chaperone has been shown to mediate reversal of heat shock-induced protein aggregation in both yeast and bacterial cells (Glover and Lindquist, Cell 94: 73-82 (1998); Parsell, et al., Nature 372: 475-478 (1994)). Therefore, since CAR D1 precipitates when expressed without a T7-derived peptide extension, it seemed possible that the T7 peptides might function by recruiting ClpB or other chaperones with similar protein refolding activity to small aggregates of CAR D1, thus mediating refolding of CAR D1. To test this model, the pET plasmid encoding the CAR D1 -T7B expression construct was transformed into an E. coli strain which had previously been deleted for the ClpB gene (Squires, et al., J. Bacteriol. 173: 4254-4262 (1991)) in order to determine whether the CAR D1-T7B fusion protein could fold into a soluble protein in the absence of functional ClpB protein. Experimental results demonstrated that the majority of the CAR D1 -T7B fusion protein was present in the soluble fraction of ClpB host cell lysates, thus indicating that the trans-acting ClpB protein chaperone does not contribute to the mechanism of T7B-mediated folding of CAR D1.

Experiments were next performed to test whether the T7B peptide mediates folding of CAR D1 by recruiting another well-characterized chaperone system which normally is induced by starvation conditions, the ssrA/SspB/ClpX system. During protein over-expression, the majority of the cell's protein synthetic capacity is redirected toward production of the over-expressed protein species, thus reducing the synthesis of endogenous host cell proteins to low levels probably similar to the levels that exist during cell starvation. Growth of bacteria under starvation conditions induces a stress response (see, Williams, et al., Mol. Microbiol. 11: 1029-1043 (1994)), in which amino acids are recovered from abortively-translated nascent polypeptides. More specifically, ribosomes “stall” during translation if aminoacyl tRNA concentrations drop below a critical level. When this occurs, a peptide-RNA complex, ssrA, loads into the vacant P site of the stalled ribosome, ultimately resulting in formation of a peptide bond between the ssrA peptide and the carboxyl-terminal end of the truncated nascent polypeptide. The ssrA tag marks the truncated polypeptide for degradation in the cell's proteasomes. See, Keiler, et al., Science 271: 990-993 (1996); Tu, et al., J. Biol. Chem. 270: 9322-9326 (1995). SsrA-mediated protein degradation requires the binding of SspB, a starvation-induced factor, to a short sequence motif in the amino-terminal half of the ssrA peptide. See, Levchenko, et al., Science 289: 2354-2356 (2000). The ClpX chaperone protein then binds to a different motif in the carboxyl-terminal half of the ssrA peptide. The resulting polypeptide-sspB-ClpX ternary complex is specifically recognized by the ClpP proteasome which then hydrolyzes the truncated polypeptide. (See, Keiler, et al., (1996); Levchenko, et al., (2000)).

The ssrA and T7 peptide extensions are similar in that both are carboxyl-terminal modifications of their substrate proteins. Additionally, the T7 peptide contains a sequence motif (AANKAR) that is similar to the SspB recognition motif in the ssrA peptide, AANDEN; where N is the dominant residue recognized by SspB. However, unlike the ssrA tag, which is always fused to truncated nascent polypeptides, the T7 peptides of the invention disclosed herein are fused to complete, full-length proteins or protein domains. Therefore, if SspB and/or ClpX recognize sequence elements in the T7 peptides, then these factors conceivably might promote folding rather than degradation of intact proteins or protein domains.

Accordingly, in order to determine whether the T7B peptide acts through a mechanism that is dependent upon binding by SspB and/or ClpX, additional mutants were constructed in which critical residues of the putative recognition sites for either SspB (i.e., Peptide T7B11 and Peptide T7B12) or ClpX (i.e., Peptide T7B9 and Peptide T7B10) were altered or deleted. Experimental results demonstrated that the yield of soluble CAR D1 was not reduced by any of these aforementioned mutations, indicating that these trans-acting factors do not contribute to the mechanism of T7B-mediated folding of CAR D1.

C. Role of peptide net charge. During analysis of T7 peptide mutants generated for the studies described above, it was observed that the partial folding-activity of peptide T7A was increased by mutation to peptide T7A1, and, conversely, that the full folding-activity of peptide T7B was reduced by mutation to peptide T7B4. The T7A1 mutant was constructed to disrupt the weak amphiphilic character of the peptide, whereas a T7B4 mutant was constructed to probe the length-dependence of the folding activity. However, as may be ascertained from Table 1, the mutation in Peptide T7A1 increases the peptide net charge from −3 to −4, whereas the Peptide T7B4 mutation decreases the peptide net charge from −6 to −2. Based on these results, additional mutants were constructed in order to systematically examine whether there was a correlation between peptide net charge and ability to mediate folding of CAR D1. As demonstrated by the experimental results not shown here, the relative proportion of soluble CAR D1 produced in E. coli increased as the net negative charge on Peptide T7A was increased from −3 to −6 (peptides T7A1, T7A2, and T7A3). Both Peptides T7A3 and T7B were found to produce almost a 100% yield of soluble CAR D1, and both species had a net negative charge of −6. Therefore, the characteristic of the carboxyl-terminal peptide extensions that is critical for their ability to mediate folding of CAR D1 appears to be the size of the net negative charge carried by the peptide extension. Consistent with this conclusion, the T3 peptide extension, which is unable to fold CAR D1, has a net charge of −2.

V. Applicability of C-Terminal Extensions to Other Test Proteins

Peptide extensions that carry a large net negative charge will significantly alter the associated protein's isoelectric point (pI). In cases where isolated domains of multidomain proteins are being expressed (as is case for the present example of CAR D1), if the isoelectric pH of the isolated domain is close to neutral, then the domain may have limited solubility in neutral pH solvents, such as the bacterial cytoplasm. Decreasing the pI of such proteins or protein fragments by attaching a peptide extension with large net negative charge may increase the solubility of these proteins or protein fragments. Since pI is an intrinsic property that varies between individual proteins, the folding-activity of a particular charged peptide extension (e.g., Peptide T7B) would be expected to vary with different protein substrates, according to this model. Alternatively, if the clustered negative charges in the peptide extension are recognized by trans-acting factors (i.e., other than ClpB, SspB, or ClpX) that promote protein folding, then a particular peptide may exhibit universal folding-activity when fused to many different proteins that normally are insoluble when over-expressed in E. coli.

In order to distinguish between these two possible mechanisms, the effect of peptide extensions on the folding of other test proteins was examined. In one experiment, the distal domain of the human A33 protein (Heath, et al., Proc. Natl. Acad. Sci. U.S.A. 94: 469-474 (1997)), the protein that is most similar to CAR D1 as revealed by homology searching using the BLAST-P program (32% identical), was examined. A33 and CAR are both members of the immunoglobulin superfamily and have similar protein and gene organization. See, Chretien, et al., Eur. J. Imunol. 28: 4094-4104 (1998). A cDNA fragment encoding the A33 distal domain (D1) was amplified by PCR and cloned into the pET15b-T7A construct in the same manner as schematically illustrated in FIG. 1 for CAR D1. When a stop codon was included to prevent fusion to the T7 peptide, the A33 protein was found to be insoluble, as was also found for CAR D1. However, unlike the results obtained with CAR D1, extending the carboxyl-terminus of A33 D1 with the T7B peptide did not increase A33 D1 solubility. Therefore, the T7B peptide does not appear to universally promote protein folding in vivo, supporting the conclusion that these peptides do not function by recruiting chaperones to the misfolded protein.

To determine if further increasing the peptide extension net negative charge would enhance folding of A33 D1, the A33 D1 domain was fused to Peptide T7B7, which has a net charge of −12 (see, Table 1). Results demonstrated that the A33 D1-T7B7 fusion protein was distributed approximately equally between the soluble and insoluble fractions of cell lysates. Only a slight further increase in fusion protein solubility resulted when A33 D1 was fused to Peptide T7B8 (data not shown), which has a net charge of −16 (see, Table 1). Because the function of A33 is unknown and consequently there is no assay for its biological activity, the A33 D1-T7B7 conformation was characterized by limited proteolysis. Staphylococcal V8 protease digested the T7B7 peptide extension more readily than the A33 D1 domain itself, as was observed for CAR D1 fusion proteins, generating digestion products which migrated with slightly faster mobility than the intact protein in SDS-PAGE. However, unlike CAR D1, the A33 D1 domain and the T7B7 peptide extension were equally sensitive to digestion with trypsin. Thus, although the A33 D1-T7B7 fusion protein is soluble, it may have a non-native conformation. This was further supported by the observation that the A33 D1-T7B7 fusion protein resolves into several species with distinct mobilities when electrophoresed under non-denaturing conditions. Together these results suggested that although the carboxyl-terminal peptide extension was able to partially solubilize A33 D1, it may not be able to mediate proper folding of the domain. Concomitant control experiments showed that both peptides T7B7 and T7B8 promote folding of CAR D1 into its biologically active conformation (data not shown), indicating that these peptides are compatible with in vivo folding of at least some proteins.

The analysis was extended to determine if the folding of other proteins could be enhanced in vivo by extending the protein C-terminus with the T7B peptide and more highly charged derivatives (T7B5-T7B8). The E. coli ClpX protein, a ˜50 kD chaperone, misfolds and aggregates into inclusion bodies when overexpressed in E. coli using pET vector technology. ClpX, therefore, is an example of how the conditions of protein overexpression can render E. coli unable to properly fold even its own endogenous proteins. As discussed above, this may result from a deficit of one or more chaperones that are required to fold nascent polypeptide chains. Fusion of the ClpX C-terminus to T7B or to T7B5-T7B8 peptides increased the fraction of the protein that was recovered in the soluble fraction of cell lysates. However, in contrast to the results obtained with A33, the C-terminal peptide extensions could be readily cleaved from the ClpX protein by limited proteolysis with both trypsin and V8 protease. Furthermore, after proteolytic removal of the T7B8-terminal extension, the resulting processed ClpX protein had full biological activity both in terms of ATPase activity and ability to cooperate with the ClpP proteasome in degrading model protein substrates.

A group of thirteen yeast proteins which are known to form inclusion bodies when over-expressed in E. coli using pET expression vectors were separately fused to the T7B peptide extension. Solubility and folding of six of these proteins was rescued to greater than 50%, while another two were rescued to a lesser extent. Solubility and folding of the remaining five proteins was not measurably affected by the T7B peptide extension (Table 2). Fusion to C-terminal peptide T7B7 failed to increase the solubility of these five refractory yeast proteins.

VI. Effect of N-Terminal Extensions on Protein Folding In Vivo

By way of example and not of limitation, one possible mechanism for the carboxyl-terminal peptide extension-mediated folding of the over-expressed proteins of the present invention is that the strong repulsive force between highly-charged peptide extensions blocks aggregation of nascent proteins. The tendency for nascent polypeptide chains to aggregate during protein overexpression could result from a deficit of chaperones, as already discussed above. If a chaperone deficit does exist during protein overexpression, then it logically follows that nascent polypeptide chains synthesized under these conditions may be more exposed to solvent than they are under normal conditions when sufficient chaperones are available to shield nascent polypeptides from solvent (cytoplasm). Just as the solubility of native proteins varies with pH of the solvent (e.g. protein solubility approaches a minimum as the pH of the solvent approaches the protein isoelectric pH), the solubility of nascent polypeptide chains that are partially or completely exposed to solvent during overexpression also may vary depending on the effective net charge of the protein species. If nascent polypeptides are exposed to solvent during their synthesis on ribosomes under conditions of overexpression, then the amount of exposed net charge also may vary as the nascent polypeptide emerges vectorially from the ribosome. According to this model, it is conceivable that unshielded nascent polypeptides may begin to precipitate co-translationally at times when the growing polypeptide chain carries little or no net charge, and that these minimally soluble species might aggregate upon release from ribosomes to form inclusion bodies. Blocking or inhibiting aggregate formation by C-terminal charged peptide extensions may provide time for the solvent-exposed, nascent polypeptide to proceed along the folding pathway and ultimately adopt the native state.

According to the above stated model, it is reasonable to expect that the solubility of nascent polypeptides also could be altered by N-terminal peptide extensions, and that this might be an alternative approach to avoiding protein aggregation in vivo. For example, if the integrated net charge of CAR D1 is plotted versus amino acid residue number (FIG. 2), one finds that the nascent polypeptide would exist as an uncharged species after synthesis of the protein was approximately 20% complete, e.g. at this point the number of positively charged and negatively charged amino acids in the growing nascent chain would be equal. If the nascent CAR D1 polypeptide is completely exposed to solvent at this point, then its solubility would be at or close to a minimum value. It is conceivable that the nascent CAR D1 polypeptide might begin to precipitate or even form small intermolecular aggregates on polyribosomes at this stage, and that these forms might be the precursors to the inclusion bodies that eventually form. However, the point at which the nascent CAR D1 polypeptide becomes an uncharged species could be altered or avoided entirely by fusion of the CAR D1 N-terminus to peptides that carry an appropriate net charge, thus avoiding co-translational precipitation of CAR D1 and the formation of inclusion bodies.

This model was tested by fusing the CAR D1 N-terminus to amino-terminal peptide extensions, according to the method outlined in FIG. 3. Consistent with the above-stated model, CAR D1 was least soluble when fused to the N-terminal peptide extensions N2 and N3 (which have neutral or +1 net charges, respectively). By contrast, CAR D1 was mostly soluble when fused to the N-terminal peptides N1 and N4, which have net charges of −2 and +2, respectively.

Results of further testing with other protein substrates were not completely consistent with this model, however. For example, the solubility of the 50 kD ClpX protein was significantly increased by fusion to the N-terminal peptide extension N2. Because the N2 peptide has no net charge, it seems unlikely that this peptide could rescue of the folding of ClpX by a mechanism dependent on peptide net charge. Rather, in this case the N-terminal peptide extension may alter the initial folding pathway of the nascent polypeptide, fortuitously avoiding the formation of folding intermediates that may precipitate or be minimally soluble under conditions of chaperone deficit. Alternatively, the N-terminal peptides may recruit chaperones to the nascent polypeptide chain.

VII. Effects of Peptide Extensions on In Vitro Renaturation

During in vitro refolding of denatured proteins, precipitation and aggregation of the protein upon removal of the denaturing agent is a common side reaction. Thus, precipitation and aggregation are problematic side reactions during the folding of proteins both in vivo and during refolding in vitro. Since carboxyl-terminal peptide extensions which carry a large net negative charge inhibit protein aggregation in vivo, possibly by increasing electrostatic charge repulsion between nascent polypeptide chains, experiments were performed to investigate whether such peptide extensions could inhibit protein aggregation during protein refolding reactions in vitro. To test this hypothesis, the A33 D1 protein fragment was produced in 2 different forms, with or without a T7B6 peptide carboxyl-terminal extension. Both forms of the A33 D1 protein were produced with an amino-terminal 6-histidine tag. When protein expression was induced at 37° C., both A33 D1 and A33 D1-T7B6 proteins misfolded and accumulated in inclusion bodies (note that A33 D1-T7B6 is only partially soluble when induction is carried out at temperatures below 25° C.).

Inclusion bodies of A33 D1 and A33 D1-T7B6 were isolated, separately, from cell lysates by differential centrifugation, and dissolved in 8 M guanidine hydrochloride (GuHCl). The solubilized proteins were then diluted with 10 volumes of renaturation buffer (10 mM Tris, 1 mM DTT, pH 8), incubated for approximately 2 hours at 4° C. to permit protein renaturation, and then dialyzed against renaturation buffer to remove the residual GuHCl denaturant. A large precipitate formed immediately upon dilution of the solubilized A33 D1 inclusion body, whereas the solubilized A33 D1-T7B6 fusion protein remained quantitatively in solution after dilution of the denaturant (the concentration of both proteins were approximately identical during the refolding reaction). After dialysis, the reactions were centrifuged to pellet the insoluble material, and the protein content of the supernatant and pellet fractions were examined by SDS-PAGE.

Experimental results (not shown) demonstrated that approximately 50% of the non-peptide-extended A33 D1 protein re-precipitated during the refolding process, whereas the peptide T7B6-extended protein was quantitatively recovered (i.e., the T7B6 peptide extension approximately doubled the recovery of soluble A33 D1). Analysis of the soluble products of the refolding reaction by electrophoresis under non-denaturing conditions showed that a small percentage of the refolded A33 D1-T7B6 migrated at a position similar to that of CAR D1, while the majority failed to migrate into the gel probably due to formation of small protein aggregates. By contrast, the refolded A33 D1 protein without the T7B6 extension appeared to migrate entirely as aggregated species.

When the soluble A33D1-T7B6 material was further analyzed by size exclusion chromatography, it was determined that the material eluted in the size range of about 100 to 200 kD, as opposed to the predicted 15 kD size. It was then discovered that the heating of the small aggregates resulted in a shift on both HPLC profiles and also non-denaturing gels, to a species of approximately 20 to 40 kD, the expected range for the native folded material. The heating conditions employed were 80° C. for 20 minutes in buffered saline.

Although at present it is not possible to definitively state whether the refolded A33 D1 has adopted its native, biologically active conformation, it can be concluded from these data that the highly charged peptide extension promotes solubility of denatured proteins following removal of the denaturing agent. Thus, the charged peptide extensions may function by a similar mechanism to promote folding of proteins in vivo and in vitro. For in vitro refolding, extension of either terminus of the protein with highly-charged peptides should introduce a strong repulsive force that promotes solubility during both chemical and heat denaturation processes.

VIII. Production of a Synthetic T7A Peptide

A synthetic peptide corresponding in sequence to peptide T7A was produced, as shown:

(acetyl-cysteine)-LEDPAANKARKEAELAAATAEQ.

An amino-terminal cysteine residue was incorporated into the peptide to introduce a reactive sulfhydryl group which could be utilized to couple the peptide to solid supports or carrier proteins.

In one set of experiments, the synthetic T7A peptide was added to in vitro protein refolding reactions to determine whether the peptide could improve yields of soluble folded protein in trans. Several different test protein systems were examined. In no case was the yield of soluble refolded protein increased by addition of the synthetic peptide (data not shown). These data support a hypothesis that the peptide extensions act to confer self-chaperoning activity to the fusion protein and that the peptides act in cis, not in trans.

In another set of experiments, lysates of E. coli strain BL2 1 -DE3 cells were passed over columns of immobilized T7A synthetic peptide (e.g. the peptide was covalently coupled to Sepharose beads via thiol linkage), to investigate whether E. coli proteins with known chaperone activity became bound to the immobilized peptide. Eluates were analyzed by Western blotting, using monoclonal antibodies specific for several different E. Coli chaperones. Eluates did not contain concentrations of chaperones that were detectable by this method, consistent with the mutagenesis studies described above, which indicated that the T7-derived peptides do not function by recruiting trans-acting chaperones.

In a final set of experiments, the synthetic T7A peptide was conjugated to KLH (keyhole limpet hemocyanin) carrier protein, emulsified in complete Freund's adjuvant and injected subcutaneously into rabbits for production of antiserum. The antiserum obtained could detect the presence of all T7-derived peptides shown in Table 1 by Western blot.

TABLE 1

| Peptide | | Net |
|---|---|---|
| Name | Sequence | Charge^{a} |
| T7C | LEDPFQSGVMLGVASTVAASPEEASVTSTEETLTPAQEAARTRAANKARKEAELAAATAEQ | −6 |
| T7B | LEDP-----------------EEASVTSTEETLTPAQEAARTRAANKARKEAELAAATAEQ | −6 |
| T7B1 | LEDP-----------------EEASVTSTEETLTPAQEAARTRAANKARKEAEL---TAEQ | −6 |
| T7B2 | LEDP-----------------EEASVTSTEETLTPAQEAARTRPPNKARKEAELAAATAEQ | −6 |
| T7B3 | LEDP-----------------EEASVTSTEETLTPAQEAARTRGGNKARKEAELAAATAEQ | −6 |
| T7B4 | LEDP-----------------------------TPAQEAARTRAANKARKEAELAAATAEQ | −2 |
| T7B5 | LEDP-----------------EEASVTSTEETLTPAQEAARTRAANKARKEAELEAETAEQ | −8 |
| T7B6 | LEDP-----------------EEASVTSTEETLTPAQEAAETEAANKARKEAELEAETAEQ | −12 |
| T7B7 | LEDP-----------------EEASVTSTEETLTPAQEAARTRAANKAEEEAELEAETAEQ | −12 |
| T7B8 | LEDP-----------------EEASVTSTEETLTPAQEAAETEAANKAEEEAELEAETAEQ | −16 |
| T7B9 | LEDP-----------------EEASVTSTEETLTPAQEAARTRAANKARKEAELAA----- | −5 |
| T7B10 | LEDP-----------------EEASVTSTEETLTPAQEAARTRAANKARKEAELAAA---- | −5 |
| T7B11 | LEDP-----------------EEASVTSTEETLTPAQEAARTRAAAKARKEAELAAATAEQ | −6 |
| T7B12 | LEDP-----------------EEASVTSTEETLTPAQEAARTR---KARKEAELAAATAEQ | −6 |
| T7B13 | LEDP-----------------EEASVTSTEETLTPAQEAARTRAANK---EAELAAATAEQ | −8 |
| T7A | LEDP---------------------------------------AANKARKEAELAAATAEQ | −3 |
| T7A1 | LEDP---------------------------------------ERNKERKEAELAAATAEQ | −4 |
| T7A2 | LEDP---------------------------------------ERNKERKEAELEAATAEQ | −5 |
| T7A3 | LEDP---------------------------------------ERNKERKEAELEAETAEQ | −6 |
| T7A4 | LEDP---------------------------------------AANKARKEAELEAATAEQ | −4 |
| T7A5 | LEDP---------------------------------------AANKARKEAELEAETAEQ | −6 |
| T3 | LEDP------------------AVWEAGKVVAKGVGTADITATTSNGLIASCKVIVNAATS | −2 |
| N1 | M-EEASVTSTEETLTPAQEAARTRAANKARKEAELAAATAEH | −2 |
| N2 | MAERASVTSTEETLTPAQEAARTRAANKARKEAELAAATAEH | 0 |
| N3 | MAEEAKVTSTEETLTPAQEAARTRAANKARKEAELAAATAEH | +1 |
| N4 | MAERAKRTSTEETLTPAQEAARTRAANKARKEAELAAATAEH | +2 |
| N5 | M-EEASVTSTEETLTPAQEAARTRAANKARKEAELEAETAEH | −4 |
| N6 | M-EEASVTSTEETLTPAQEAAETEAANKARKEAELEAETAEH | −8 |
| N7 | M-EEASVTSTEETLTPAQEAARTRAANKAEEEAELEAETAEH | −8 |

^{a}The terminal COO⁻ and NH₃⁺ groups of carboxyl-terminal and amino terminal peptide extensions were included in the calculation of peptide net charge

TABLE 2
Effect of T7B carboxyl-terminal peptide extension on the folding
of yeast proteins

| SwissProt | Protein size | Improvement | Improvement |
|---|---|---|---|
| Access # (^{a}) | (# of amino acids) | 37° C.^{b} | 25° C.^{b} |
| P0-6633 (1) | 220 | 85% | 85% |
| P40099 (9) | 210 | none | none |
| P40961 (55) | 287 | none | none |
| P46948 (56) | 246 | none | 50% |
| P18562 (60) | 251 | none | 30% |
| P40530 (65) | 394 | none | none |
| P47076 (67) | 161 | 10% | 70% |
| P06838 (84) | 210 | none | none |
| Q03219 (90) | 274 | 50% | 50% |
| P53889 (96) | 259 | 50% | 70% |
| P53727 (99) | 317 | none | 10% |
| P06174 (106) | 275 | none | none |
| Q02784 (107) | 150 | 50% | 70% |

^{a}numbers in parentheses are ID numbers assigned to yeast proteins selected for analysis by the Brookhaven National Laboratory Proteomics Projects (see http://proteome.bnl.gov/progress.html).
^{b}estimated increase in the recovery of protein in the soluble fraction of cell lystates, as compared to the yield of the unmodified protein expressed under similar conditions.

1. A method for enhancing the solubility of, and promoting the adoption of native folding conformation, of a protein or polypeptide expressed by recombinant DNA techniques in a host cell, the method comprising:
a) providing a first nucleic acid sequence encoding a protein or polypeptide of interest, the protein or polypeptide being substantially insoluble, or biologically inactive, when expressed in a host cell by recombinant DNA techniques;
b) providing a second nucleic acid sequence encoding a peptide extension having a net negative charge, the peptide T7A of Table 1 being specifically excluded;
c) fusing the second nucleic acid sequence to the first nucleic acid sequence in an expression vector such that a fusion protein encoded by the first and second nucleic acid sequences is expressed in the host cell following transformation of the host cell with the expression vector encoding the fusion protein, the peptide extension encoded by the second nucleic acid sequence being positioned at the carboxyl-terminus of the protein or polypeptide of interest;
d) transforming the host cell with the expression vector encoding the fusion protein; and
e) culturing the transformed host cells under conditions appropriate for the expression of the fusion protein.

2. The method of claim 1 wherein the host cell is a prokaryotic cell.

3. The method of claim 1 wherein the host cell is a eukaryotic cell.

4. The method of claim 1, wherein the net negative charge of the peptide extension ranges from −2 to −20.

5. The method of claim 1, wherein the net negative charge of the peptide extension is from −15 to −20.

6. The method of claim 1, wherein the net negative charge of the peptide extension is from −10 to −14.

7. The method of claim 1, wherein the net negative charge of the peptide extension is from −5 to −9.

8. The method of claim 1, wherein the net negative charge of the peptide extension is from −2 to −4.

9. The method of claim 1, wherein the peptide extension adopts a non-ordered conformation following expression.

10. The method of claim 1 wherein the peptide extension comprises about 61 amino acid residues or less.

11. The method of claim 1, wherein the peptide extension comprises the 57 residue carboxyl-terminal portion of the T7 gene 10B protein, or solubility or activity promoting portions thereof.

12. The method of claim 11 wherein the peptide extension further comprises amino acid substituted variants of the 57 residue carboxyl terminal portion of the T7 gene 10B protein, or active portions thereof, which modifications result in the maintenance of a net negative charge of between −2 and −20.

13. The method of claim 11, wherein the peptide extension is selected from the group consisting of: Peptide T7C, Peptide T7B, Peptide T7B1, Peptide T7B2, Peptide T7B3, Peptide T7B5, Peptide T7B6, Peptide T7B7, Peptide T7B8, Peptide T7B9, Peptide T7B10, Peptide T7B11, Peptide T7B12, Peptide T7B13, Peptide T7A1, Peptide T7A2, Peptide T7A3, Peptide T7A4, and Peptide T7A5.

14. A method for enhancing the solubility, and promoting the adoption of native folding conformation, of a protein or polypeptide expressed by recombinant DNA techniques in a host cell, the method comprising:
a) providing a first nucleic acid sequence encoding a protein or polypeptide of interest, the protein or polypeptide being substantially insoluble, or biologically inactive, when expressed in a host cell by recombinant DNA techniques;
b) providing a second nucleic acid sequence encoding a peptide extension having a net charge ranging from +2 to −20;
c) fusing the second nucleic acid sequence to the first nucleic acid sequence in an expression vector such that a fusion protein encoded by the first and second nucleic acid sequences is expressed in the host cell following transformation of the host cell with the expression vector encoding the fusion protein, the peptide extension encoded by the second nucleic acid sequence being positioned at the amino-terminus of the protein or polypeptide of interest;
d) transforming the host cell with the expression vector encoding the fusion protein, under conditions appropriate for expression of the fusion protein; and
e) culturing the transformed host cells under conditions appropriate for the expression of the fusion protein.

15. The method of claim 14 wherein the host cell is a prokaryotic cell.

16. The method of claim 14 wherein the host cell is a eukaryotic cell.

17. The method of claim 14, wherein the net charge of the peptide extension is from −15 to −20.

18. The method of claim 14, wherein the net charge of the peptide extension is from −10 to −14.

19. The method of claim 14, wherein the net charge of the peptide extension is from −5 to −9.

20. The method of claim 14, wherein the net charge of the peptide extension is from −1 to −4.

21. The method of claim 14, wherein the net charge of the peptide extension is from +2 to −1.

22. The method of claim 14, wherein the peptide extension adopts a non-ordered conformation following expression.

23. The method of claim 14, wherein the peptide extension comprises the 57 residue carboxyl-terminal portion of the T7 gene 10B protein, or solubility or activity promoting portions thereof.

24. The method of claim 23 wherein the peptide extension further comprises amino acid substituted variants of the 57 residue carboxyl terminal portion of the T7 gene 10B protein, or active portions thereof, which modifications result in the maintenance of a net charge of between +2 and −20.

25. The method of claim 23, wherein the peptide extension is selected from the group consisting of: Peptide N1, Peptide N2, Peptide N3, Peptide N4, Peptide N5, Peptide N6, and Peptide N7.

26. A method for enhancing the in vitro renaturation of a protein or polypeptide expressed by recombinant DNA techniques in a host cell, a substantial percentage of the expressed protein or polypeptide being localized in inclusion bodies following expression in the host cell, the method comprising:
a) providing a first nucleic acid sequence encoding a protein or polypeptide of interest;
b) providing a second nucleic acid sequence encoding a peptide extension having a net negative charge, the peptide T7A of Table 1 being specifically excluded;
c) fusing the second nucleic acid sequence to the first nucleic acid sequence in an expression vector such that a fusion protein encoded by the first and second nucleic acid sequences is expressed in a host cell following transformation of the host cell with the expression vector encoding the fusion protein, the peptide extension encoded by the second nucleic acid sequence being positioned at the carboxyl-terminus of the protein or polypeptide of interest;
d) transforming the host cell with the expression vector encoding the fusion protein, under conditions appropriate for expression of the fusion protein;
e) isolating inclusion bodies from lysates of the host cell;
f) contacting the isolated inclusion bodies with a denaturing solution thereby solubilizing the fusion protein comprising the inclusion body; and,
g) suspending the solubilized fusion protein of step f) in a renaturation buffer.

27. The method of claim 26 wherein the host cell is a prokaryotic cell.

28. The method of claim 26 wherein the host cell is a eukaryotic cell.

29. The method of claim 26 further comprising a heat denaturation step.

30. The method of claim 26, wherein the net negative charge of the peptide extension ranges from −2 to −20.

31. The method of claim 26, wherein the net negative charge of the peptide extension is from −15 to −20.

32. The method of claim 26, wherein the net negative charge of the peptide extension is from −10 to −14.

33. The method of claim 26, wherein the net negative charge of the peptide extension is from −5 to −9.

34. The method of claim 26, wherein the net negative charge of the peptide extension is from −1 to −4.

35. The method of claim 26, wherein the peptide extension adopts a non-ordered conformation following expression.

36. The method of claim 26 wherein the peptide extension comprises about 61 amino acid residues or less.

37. The method of claim 26, wherein the peptide extension comprises the 57 residue carboxyl-terminal portion of the T7 gene 10B protein, or solubility or activity promoting portions thereof.

38. The method of claim 37 wherein the peptide extension further comprises amino acid substituted variants of the 57 residue carboxyl terminal portion of the T7 gene 10B protein, or active portions thereof, which modifications result in the maintenance of a net negative charge of between −2 and −20.

39. The method of claim 37, wherein the peptide extension is selected from the group consisting of: Peptide T7C, Peptide T7B, Peptide T7B1, Peptide T7B2, Peptide T7B3, Peptide T7B5, Peptide T7B6, Peptide T7B7, Peptide T7B8, Peptide T7B9, Peptide T7B10, Peptide T7B11, Peptide T7B12, Peptide T7B13, Peptide T7A1, Peptide T7A2, Peptide T7A3, Peptide T7A4, and Peptide T7A5.

40. A method for enhancing the in vitro renaturation of a protein or polypeptide expressed by recombinant DNA techniques in a host cell, a substantial percentage of the expressed protein or polypeptide being localized in inclusion bodies following expression in the host cell, the method comprising:
a) providing a first nucleic acid sequence encoding a protein or polypeptide of interest;
b) providing a second nucleic acid sequence encoding a peptide extension having a net charge ranging from +2 to −20;
c) fusing the second nucleic acid sequence to the first nucleic acid sequence in an expression vector such that a fusion protein encoded by the first and second nucleic acid sequences is expressed in a host cell following transformation of the host cell with the expression vector encoding the fusion protein, the peptide extension encoded by the second nucleic acid sequence being positioned at the amino-terminus of the protein or polypeptide of interest;
d) transforming the host cell with the expression vector encoding the fusion protein, under conditions appropriate for expression of the fusion protein;
e) isolating inclusion bodies from lysates of the host cell;
f) contacting the isolated inclusion bodies with a denaturing solution thereby solubilizing the fusion protein comprising the inclusion body; and
g) suspending the solubilized fusion protein of step f) in a renaturation buffer.

41. The method of claim 40 wherein the host cell is a prokaryotic cell.

42. The method of claim 40 wherein the host cell is a eukaryotic cell.

43. The method of claim 40 further comprising a heat denaturation step.

44. The method of claim 40, wherein the net charge of the peptide extension is from −15 to −20.

45. The method of claim 40, wherein the net charge of the peptide extension is from −10 to −14.

46. The method of claim 40, wherein the net charge of the peptide extension is from −5 to −9.

47. The method of claim 40, wherein the net charge of the peptide extension is from −1 to −4.

48. The method of claim 40, wherein the net charge of the peptide extension is from +2 to −1.

49. The method of claim 40, wherein the peptide extension adopts a non-ordered conformation following expression.

50. The method of claim 40, wherein the peptide extension comprises the 57 residue carboxyl-terminal portion of the T7 gene 10B protein, or solubility or activity promoting portions thereof.

51. The method of claim 50 wherein the peptide extension further comprises amino acid substituted variants of the 57 residue carboxyl terminal portion of the T7 gene 10B protein, or active portions thereof, which modifications result in the maintenance of a net charge of between +2 and −20.

52. The method of claim 50, wherein the peptide extension is selected from the group consisting of: Peptide N1, Peptide N2, Peptide N3, Peptide N4, Peptide N5, Peptide N6, and Peptide N7.

53. An expression vector comprising a nucleic acid sequence encoding a peptide extension, the peptide extension having a net negative charge ranging from −2 to −20; the expression vector comprising a multiple cloning site for inserting, in-frame with said peptide extension, a nucleic acid sequence encoding a protein or polypeptide of interest, wherein the expression of the nucleic acid sequences yields a fusion protein in which the peptide extension is fused to the carboxyl-terminus of the protein or polypeptide of interest.

54. The vector of claim 53 which is optimized for use with a prokaryotic cell.

55. The vector of claim 53 which is optimized for use with a eukaryotic cell.

56. The expression vector of claim 53, wherein the net charge of the peptide extension is from −15 to −20.

57. The expression vector of claim 53, wherein the net charge of the peptide extension is from −10 to −14.

58. The expression vector of claim 53, wherein the net charge of the peptide extension is from −5 to −9.

59. The expression vector of claim 53, wherein the net charge of the peptide extension is from −2 to −4.

60. The expression vector of claim 53, wherein the peptide extension adopts a non-ordered conformation following expression.

61. The expression vector of claim 53 wherein the peptide extension comprises about 61 amino acid residues or less.

62. The expression vector of claim 53, wherein the peptide extension comprises the 57 residue carboxyl-terminal portion of the T7 gene 10B protein, or solubility or activity promoting portions thereof.

63. The expression vector of claim 62 wherein the peptide extension further comprises amino acid substituted variants of the 57 residue carboxyl terminal portion of the T7 gene 10B protein, or active portions thereof, which modifications result in the maintenance of a net negative charge of between −2 and −20.

64. The expression vector of claim 62, wherein the peptide extension is selected from the group consisting of: Peptide T7C, Peptide T7B, Peptide T7B1, Peptide T7B2, Peptide T7B3, Peptide T7B5, Peptide T7B6, Peptide T7B7, Peptide T7B8, Peptide T7B9, Peptide T7B10, Peptide T7B11, Peptide T7B12, Peptide T7B13, Peptide T7A1, Peptide T7A2, Peptide T7A3, Peptide T7A4, and Peptide T7A5.

65. An expression vector comprising a nucleic acid sequence encoding a peptide extension, the peptide extension having a net charge ranging from +2 to −20; the expression vector comprising a multiple cloning site for inserting, in-frame with said peptide extension, a nucleic acid sequence encoding a protein or polypeptide of interest, wherein the expression of the nucleic acid sequences yields a fusion protein in which the peptide extension is fused to the amino-terminus of the protein or polypeptide of interest.

66. The vector of claim 65 which is optimized for use with a prokaryotic cell.

67. The vector of claim 65 which is optimized for use with a eukaryotic cell.

68. The expression vector of claim 65, wherein the net charge of the peptide extension is from −15 to −20.

69. The expression vector of claim 65, wherein the net charge of the peptide extension is from −10 to −14.

70. The expression vector of claim 65, wherein the net charge of the peptide extension is from −5 to −9.

71. The expression vector of claim 65, wherein the net charge of the peptide extension is from +2 to −4.

72. The expression vector of claim 65, wherein the peptide extension adopts a non-ordered conformation following expression.

73. The expression vector of claim 65 wherein the peptide extension comprises about 61 amino acid residues or less.

74. The expression vector of claim 65, wherein the peptide extension comprises the 57 residue carboxyl-terminal portion of the T7 gene 10B protein, or solubility or activity promoting portions thereof.

75. The expression vector of claim 74 wherein the peptide extension further comprises amino acid substituted variants of the 57 residue carboxyl terminal portion of the T7 gene 10B protein, or active portions thereof, which modifications result in the maintenance of a net charge of between +2 and −20.

76. The expression vector of claim 74, wherein the peptide extension is selected from the group consisting of: Peptide N1, Peptide N2, Peptide N3, Peptide N4, Peptide N5, Peptide N6, and Peptide N7.

77. A method for enhancing the solubility and promoting the adoption of native folding conformation of a recombinant protein or polypeptide of interest, which protein or polypeptide would otherwise adopt a non-native conformation and form an insoluble inclusion body when expressed by recombinant DNA techniques in a host cell, the method comprising expressing said protein or polypeptide as a fusion protein wherein the protein or polypeptide is fused to a charged peptide extension, said peptide extension comprising 61 amino acid residues or less and which peptide extension confers a self-chaperoning activity to the fusion protein.

78. The method of claim 77, wherein the peptide extension is fused to the carboxyl-terminus of the protein or polypeptide of interest.

79. The method of claim 77, wherein the peptide extension is fused to the amino-terminus of the protein or polypeptide of interest.

80. A method for enhancing the solubility of, and promoting the adoption of native folding conformation, of a protein or polypeptide expressed by recombinant DNA techniques in a prokaryotic cell, the method comprising:
a) providing a first nucleic acid sequence encoding a protein or polypeptide of interest, the protein or polypeptide being substantially insoluble, or biologically inactive, when expressed in a prokaryotic cell by recombinant DNA techniques;
b) providing a second nucleic acid sequence encoding a peptide extension having a net negative charge, the peptide T7A of Table 1 being specifically excluded;
c) fusing the second nucleic acid sequence to the first nucleic acid sequence in a prokaryotic expression vector such that a fusion protein encoded by the first and second nucleic acid sequences is expressed in a prokaryotic cell following transformation of the prokaryotic cell with the prokaryotic expression vector encoding the fusion protein, the peptide extension encoded by the second nucleic acid sequence being positioned at the carboxyl-terminus of the protein or polypeptide of interest;
d) transforming the prokaryotic cell with the prokaryotic expression vector encoding the fusion protein; and
e) culturing the transformed prokaryotic cells under conditions appropriate for the expression of the fusion protein.

81. A method for enhancing the solubility, and promoting the adoption of native folding conformation, of a protein or polypeptide expressed by recombinant DNA techniques in a prokaryotic cell, the method comprising:
a) providing a first nucleic acid sequence encoding a protein or polypeptide of interest, the protein or polypeptide being substantially insoluble, or biologically inactive, when expressed in a prokaryotic cell by recombinant DNA techniques;
b) providing a second nucleic acid sequence encoding a peptide extension having a net charge ranging from +2 to −20;
c) fusing the second nucleic acid sequence to the first nucleic acid sequence in a prokaryotic expression vector such that a fusion protein encoded by the first and second nucleic acid sequences is expressed in a prokaryotic cell following transformation of the prokaryotic cell with the prokaryotic expression vector encoding the fusion protein, the peptide extension encoded by the second nucleic acid sequence being positioned at the amino-terminus of the protein or polypeptide of interest;
d) transforming the prokaryotic cell with the prokaryotic expression vector encoding the fusion protein, under conditions appropriate for expression of the fusion protein; and
e) culturing the transformed prokaryotic cells under conditions appropriate for the expression of the fusion protein.

82. A method for enhancing the in vitro renaturation of a protein or polypeptide expressed by recombinant DNA techniques in a prokaryotic cell, a substantial percentage of the expressed protein or polypeptide being localized in inclusion bodies following expression in the prokaryotic cell, the method comprising:
a) providing a first nucleic acid sequence encoding a protein or polypeptide of interest;
b) providing a second nucleic acid sequence encoding a peptide extension having a net negative charge, the peptide T7A of Table 1 being specifically excluded;
c) fusing the second nucleic acid sequence to the first nucleic acid sequence in a prokaryotic expression vector such that a fusion protein encoded by the first and second nucleic acid sequences is expressed in a prokaryotic cell following transformation of the prokaryotic cell with the prokaryotic expression vector encoding the fusion protein, the peptide extension encoded by the second nucleic acid sequence being positioned at the carboxyl-terminus of the protein or polypeptide of interest;
d) transforming the prokaryotic cell with the prokaryotic expression vector encoding the fusion protein, under conditions appropriate for expression of the fusion protein;
e) isolating inclusion bodies from lysates of the prokaryotic cell;
f) contacting the isolated inclusion bodies with a denaturing solution thereby solubilizing the fusion protein comprising the inclusion body; and,
g) suspending the solubilized fusion protein of step f) in a renaturation buffer.

83. A method for enhancing the in vitro renaturation of a protein or polypeptide expressed by recombinant DNA techniques in a prokaryotic cell, a substantial percentage of the expressed protein or polypeptide being localized in inclusion bodies following expression in the prokaryotic cell, the method comprising:
a) providing a first nucleic acid sequence encoding a protein or polypeptide of interest;
b) providing a second nucleic acid sequence encoding a peptide extension having a net charge ranging from +2 to −20;
c) fusing the second nucleic acid sequence to the first nucleic acid sequence in a prokaryotic expression vector such that a fusion protein encoded by the first and second nucleic acid sequences is expressed in a prokaryotic cell following transformation of the prokaryotic cell with the prokaryotic expression vector encoding the fusion protein, the peptide extension encoded by the second nucleic acid sequence being positioned at the amino-terminus of the protein or polypeptide of interest;
d) transforming the prokaryotic cell with the prokaryotic expression vector encoding the fusion protein, under conditions appropriate for expression of the fusion protein;
e) isolating inclusion bodies from lysates of the prokaryotic cell;
f) contacting the isolated inclusion bodies with a denaturing solution thereby solubilizing the fusion protein comprising the inclusion body; and
g) suspending the solubilized fusion protein of step f) in a renaturation buffer.

84. An antibody which binds specifically to one or more polypeptides selected from the group consisting of: Peptide T7C, Peptide T7B, Peptide T7B1, Peptide T7B2, Peptide T7B3, Peptide T7B4, Peptide T7B5, Peptide T7B6, Peptide T7B7, Peptide T7B8, Peptide T7B9, Peptide T7B10, Peptide T7B11, Peptide T7B12, Peptide T7B13, Peptide T7A, Peptide T7A1, Peptide T7A2, Peptide T7A3, Peptide T7A4, Peptide T7A5, N1, N2, N3, N4, N5, N6, and N7 described in Table 1.

85. The antibody of claim 84 which is monoclonal.

86. The antibody of claim 84 which is polyclonal.
